(19) United States
(12) Patent Application Publication
(10) Pub. No.: US 2012/0302996 A1
(43) Pub. Date: Nov. 29, 2012

(54) BALLOON CATHETER AND METHODS OF USE THEREOF

(76) Inventors: Alexander Barash, Tzoran (IL); Roy Feig, Jerusalem (IL); Eran Harari, Maagan Michael (IL)

(21) Appl. No.: 13/497,055

(22) PCT Filed: Oct. 3, 2010
(86) PCT No.: PCT/IL10/00797

§ 371 Date: Aug. 2, 2012

Related U.S. Application Data
(60) Provisional application No. 61/245,155, filed on Sep. 23, 2009.

(51) Int. Cl. A61M 25/10

(52) U.S. Cl. 604/509; 604/103.04; 604/99.04

(57) ABSTRACT

A rapid exchange catheter has an outer conduit and a hollow inner conduit disposed in the lumen of the outer conduit. The inner conduit includes a distal part, a proximal part and a variable length sleeve member sealingly attached between the proximal part and distal part. The distal end of the distal part of the inner conduit extends beyond the distal end of the outer conduit. An inflatable balloon is sealingly attached to the distal end of the outer conduit and to the proximal part of the inner conduit. The balloon intussuscepts upon proximal movement of the distal part of the inner conduit. A pulling member is attached to the distal part of the inner conduit for moving the distal part of the inner conduit proximally to longitudinally shorten the sleeve member. A fluid port allows inflating and deflating the balloon.

### FIELD OF THE INVENTION

This invention relates in general to the field of medical catheters having inflatable balloons and more particularly to rapid-exchange catheters having an intussuscepting balloon.

### BACKGROUND OF THE INVENTION

Catheters are used in various interventional procedures for delivering therapeutic means to a treated site (e.g., body organ or passageway such as blood vessels). In many cases, a catheter with a small distal inflatable balloon is guided to the treated site. Once the balloon is in place it is inflated by the operator for affixing it in place, for expanding a blocked vessel, for placing treatment means (e.g., stent) and/or for delivering surgical tools (e.g. knives, drills etc.) to a desired site. In addition, catheter systems have also been designed and used for retrieval of objects such as stents from body passageways.

Rapid-exchange catheters have been developed for intravascular use and are routinely used for anngioplastic treatment of stenosed vessels in patients.

Rapid exchange (“monorail”) catheters typically comprise a relatively short guide wire lumen provided in a distal section thereof, and a proximal guide wire exit port located between the catheter's distal and proximal ends. This arrangement allows exchange of the catheter over a relatively short guide wire, in a manner which is simple to perform and which can be carried out by a single operator. Rapid exchange catheters have been extensively described in the art, for example, in U.S. Pat. Nos. 4,762,129, 4,748,982 and EP0380873.

Rapid exchange catheters are commonly used in Percutaneous Transluminal Coronary Angioplasty (PTCA) procedures, in which obstructed blood vessels are typically dilated by a distal balloon mounted on the catheter's distal end. A stent is often placed at the vessel's dilation zone to prevent reoccurrences of obstruction therein. The dilation balloon is typically inflated via an inflation lumen which extends longitudinally inside the catheter's shaft between the dilation balloon and the catheter's proximal end.

Published International Patent Application, Publication No. WO 2005/102184 discloses a catheter having a rollable expandable element. Published International Patent applications, Publication Nos. WO 2007/004221, WO 2007/042935, WO 2008/004238 and WO 2008/004239, all five published international applications are incorporated herein by reference in their entirety for all purposes, disclose various types of catheters and catheter systems having intussuscepting balloon-like inflatable members which may be used, inter alia, to treat plaque by balloon inflation while efficiently and safely collecting plaque debris and other particulate matter from the lumen of pathologically-involved blood vessels and to remove such particles and particulate matter from the blood vessel.

WO 2008/004238 discloses several types of rapid exchange catheters having an intussuscepting balloon-like inflatable member which may be used for treating plaque in stenosed vessels and for collecting and for removing from the body plaque debris and other particulate matter resulting from the distention of the vessel wall and the compaction of plaque during the inflating of the balloon within the blood vessel.

While the various types of rapid exchange catheters with intussuscepting balloons disclosed in WO 2008/004238 may be efficiently and safely used for treating patients, their construction is based on the use of a segmented tubular inner conduit having several segments. Some segments of the inner conduit are slidably disposed within other segments of the inner conduit in order to enable the distal part of the inner conduit to move proximally during the intussuscepting of the balloon. In order to keep the segmented inner conduit sealed, WO 2008/004238 teaches the use of sealing gaskets designed to withstand the inflation pressure of the balloons. While sealing gaskets are well known in the art their use may pose several technical difficulties, due mainly to the fact that the implementation of sealing gaskets may require expensive and time consuming construction techniques as well as the use of time consuming and expensive testing and quality control procedures. This is especially challenging when the diameters of inner conduit and of the necessary gaskets are relatively small.

### SUMMARY OF THE INVENTION

There is therefore provided, in accordance with an embodiment of the catheters of the present application, a rapid exchange balloon catheter. The catheter includes an outer conduit having a distal end and a proximal end. The catheter also includes a hollow inner conduit, suitable for passage over a guide wire. The inner conduit includes a distal part, a proximal part and a variable length sleeve member sealingly attached between the proximal part and the distal part of the inner conduit. The proximal part of the inner conduit is sealingly attached to the distal end of the outer conduit. The inner conduit is disposed within the lumen of the outer conduit such that the distal end of the distal part of the inner conduit extends beyond the distal end of the outer conduit. The catheter also includes a pulling member having a distal end attached to the distal part of the inner conduit and a proximal end disposed outside of the outer conduit. The pulling member is movably disposed within the catheter such that when the pulling member is moved proximally, the distal part of the inner conduit moves proximally to longitudinally shorten the sleeve member. The catheter also includes an inflatable balloon having a proximal balloon end sealingly attached to the outer surface of the distal end of the outer conduit, and a distal balloon end sealingly attached to the outer surface of the proximal part of the inner conduit. The distal end of the balloon is capable of intussuscepting upon proximal movement of the distal part of the inner conduit in relation to the outer conduit. The catheter also includes a fluid port for the introduction of an inflation fluid into the space formed between the inner surface of the outer conduit and the outer surface of the inner conduit and therefrom into the lumen of the balloon and for removal of the inflation fluid from the balloon.

There is also provided, in accordance with an embodiment of the catheters of the present application, a rapid exchange balloon catheter. The catheter includes an outer conduit having a distal end and a proximal end. The catheter also includes a hollow inner conduit, suitable for passage over a guide wire. The inner conduit includes a distal part, a proximal part and a variable length sleeve member sealingly attached between the proximal part and the distal part of the inner conduit. The proximal part of the inner conduit is sealingly attached to the distal end of the outer conduit. The inner conduit is disposed within the lumen of the outer conduit and positioned such that the distal end of the distal part of the inner conduit extends beyond the distal end of the outer conduit. The catheter also includes pulling/pushing means for proximally pulling and for distally pushing the distal part of the inner conduit within the outer conduit, such that when the pulling/pushing means is pulled proximally, the distal part of the inner conduit moves proximally to longitudinally shorten the sleeve member. The catheter also includes an inflatable balloon having a proximal balloon end sealingly attached to the outer surface of the distal end of the outer conduit, and a distal balloon end sealingly attached to the outer surface of the proximal part of the inner conduit. The distal end of the balloon is capable of intussuscepting upon proximal movement of the distal part of the inner conduit in relation to the outer conduit. The catheter also includes means for introducing an inflation fluid into the catheter and the balloon and for removal of the inflation fluid from the catheter and from the balloon.

Furthermore, in accordance with an embodiment of the catheters of the present application, the catheter according also includes a pressure controlling mechanism for preventing or attenuating pressure changes within the balloon upon intussuscepting of the balloon.

Furthermore, in accordance with an embodiment of the catheters of the present application, the pressure controlling mechanism is selected from, a syringe-like member in fluidic communication with the balloon, a hydraulic accumulator in fluidic communication with the balloon, a pressure relief valve in fluidic communication with the balloon, and any combinations thereof. The syringe-like member includes a plunger member disposed therein. The plunger is attached to the pulling member such that when the plunger member is pulled proximally, the pulling member moves proximally within the catheter to cause intussuscepting of the balloon. At least some of the inflation fluid ejected from the balloon is accommodated within a volume formed within the syringe like member upon proximal pulling of the plunger member.

Furthermore, in accordance with an embodiment of the catheters of the present application the pressure relief valve is an overpressure valve adapted to discharge inflation fluid whenever the pressure within the catheter exceeds a threshold pressure value.

Furthermore, in accordance with an embodiment of the catheters of the present application, the variable length sleeve member is selected from, a corrugated sleeve like member, a cylindrical sleeve like member having a circular cross section, a sleeve like member having a non-circular cross section, and a sleeve like member having at least one corrugated portion and at least one non-corrugated portion.

Furthermore, in accordance with an embodiment of the catheters of the present application, the length of the variable length sleeve member in the fully extended state is selected from a length smaller than half the length of the balloon when the balloon is fully extended, a length equal to half the length of the balloon when the balloon is fully extended, and a length larger than half the length of the balloon when the balloon is fully extended.

Furthermore, in accordance with an embodiment of the catheters of the present application, the balloon is selected from, a corrugated balloon, a balloon having at least one corrugated portion, a stepped balloon, a conical balloon, a distally tapering balloon, a distally and proximally tapering balloon, a balloon having a non-uniform wall thickness, a balloon having a larger balloon wall thickness on its proximal portion, a balloon having a reinforced proximal portion, a stepped balloon having one or more corrugated parts, a balloons having a thickened proximal balloon part, a balloon having a rounded distal end, a balloon having one or more tapered parts, a balloon having one or more conical parts, and a balloon having one or more frusto-conical parts.

Furthermore, in accordance with an embodiment of the catheters of the present application, the balloon is selected from, a balloon having in its inflated state, a shape which is capable of guiding the intussuscepting of the distal portion of said balloon upon proximal movement of said distal part of said inner conduit in relation to the outer conduit, a balloon having, in its inflated state, a distal taper with a rounded distal extremity, and a balloon having, in its inflated state, a proximal taper with a rounded proximal extremity.

Furthermore, in accordance with an embodiment of the catheters of the present application, the proximal part of the inner conduit is selected from, a straight proximal part sealingly attached to and opening at the proximal end of the outer conduit, and a laterally curved proximal part sealingly attached to and opening at the lateral side of the outer conduit.

There is also provided, in accordance with an embodiment of the methods of the present application, a method for collecting debris from an internal passage of a mammalian subject. The method includes the steps of:
(a) inserting any of rapid exchange balloon catheter embodiments disclosed hereinabove into the internal passage, and advancing the catheter until the distal end thereof has reached a target site, at which it is desired to collect debris,
(b) inflating the balloon with inflation fluid,
(c) moving the distal part of the inner conduit in a proximal direction, to cause intussuscepting of the distal end of the balloon,
(d) deflating the balloon, to form therein a cavity into which debris is collected and entrapped, and
(e) removing the catheter from the internal passage of the subject, together with the entrapped debris.

Furthermore, in accordance with an embodiment of the methods of the present application, the internal passage is a blood vessel.

Furthermore, in accordance with an embodiment of the methods of the present application, the target site is in the vicinity of a stenosed portion of the blood vessel and the method also includes the step of introducing through the lumen of the inner conduit of the castheter a medical device for treating the stenosed portion.

Furthermore, in accordance with an embodiment of the methods of the present application, said medical device is selected from a diagnostic device, a treatment device, and a combined treatment device and diagnostic device.

Furthermore, in accordance with an embodiment of the methods of the present application, the treatment device is selected from an mechanical atherectomy device, a laser atherectomy device, an ultrasonic treatment device, an embolic protection device, a filter device, a basket-like device, a blade-like device, an occluding balloon device, an aspirating device and any combinations thereof.

Furthermore, in accordance with an embodiment of the methods of the present application, the diagnostic device is selected from an imaging device, an infra-red imaging device, an imaging device operating in the visual range of the electromagnetic radiation spectrum, an ultrasonic imaging device, a magnetic resonance imaging device, an electrode based sensing device, a temperature sensing device, an electrochemical sensing device, a device for sensing the concentration of a chemical species and any combinations thereof.

Furthermore, in accordance with an embodiment of the methods of the present application, the step of introducing a medical device also includes treating the blood vessel using the medical device.

Furthermore, in accordance with an embodiment of the methods of the present application, the step of introducing a medical device also includes performing a diagnostic procedure on at least a portion of the blood vessel using the medical device.

Furthermore, in accordance with an embodiment of the methods of the present application, the method further includes the step of withdrawing the medical device outside of the catheter through the lumen of the inner conduit prior to performing step (c).

Furthermore, in accordance with an embodiment of the methods of the present application, the method further includes the step, of withdrawing the medical device into the lumen of the inner conduit prior to performing step (e).

Furthermore, in accordance with an embodiment of the methods of the present application, the method further includes the step of introducing a stent into said body passage using said balloon.

Finally, in accordance with an embodiment of the methods of the present application, the method further includes the step of deploying said stent within said body passage during said inflating of step (b).

### BRIEF DESCRIPTION OF THE DRAWINGS

The invention is herein described, by way of example only, with reference to the accompanying drawings, in which like components are designated by like reference numerals, wherein:

FIG. 1 is a schematic cross-sectional diagram illustrating a rapid-exchange intussuscepting balloon catheter having an inner conduit comprising a proximal part sealingly connected to a distal part by a variable length sleeve member, in accordance with an embodiment of the catheter of the present application;

FIG. 2 is a schematic cross-sectional diagram illustrating the rapid exchange catheter of FIG. 1 with the balloon in an inflated and intussuscepted state achieved by proximally pulling a pulling member attached to the distal part of the inner conduit and causing the thin flexible part of the inner conduit to longitudinally shorten, in accordance with an embodiment of the catheter of the present application;

FIG. 3. is a schematic cross-sectional diagram illustrating the rapid exchange catheter of FIGS. 1-2 with the balloon being in an intussuscepted and deflated state;

FIG. 4 is a schematic cross-sectional diagram illustrating a rapid-exchange intussuscepting balloon catheter having a handle including a pressure controlling mechanism including a pressure relief valve, in accordance with an embodiment of the catheter of the present application; and

FIG. 5 is a schematic cross-sectional diagram illustrating a rapid-exchange intussuscepting balloon catheter having a pressure controlling mechanism including a hydraulic accumulator, in accordance with an embodiment of the catheter of the present application; and

FIG. 6 is a schematic cross-sectional diagram illustrating a rapid exchange intussuscepting balloon catheter having a curved inner conduit having a lumen that opens on the lateral side of the outer conduit of the catheter, in accordance with another embodiment of the catheters of the present application.

### DETAILED DESCRIPTION OF THE INVENTION

The present application discloses a rapid exchange intussuscepting balloon catheter having a gasket-free inner conduit.

It is noted that the drawing figures are not drawn to scale, are schematic, and are for illustrative purposes only, it should therefore be appreciated that the illustrated dimensions of any of the drawings are not representative of the true dimensions and that the relationship of the dimensions of different components and parts relative to each other are not necessarily accurately drawn.

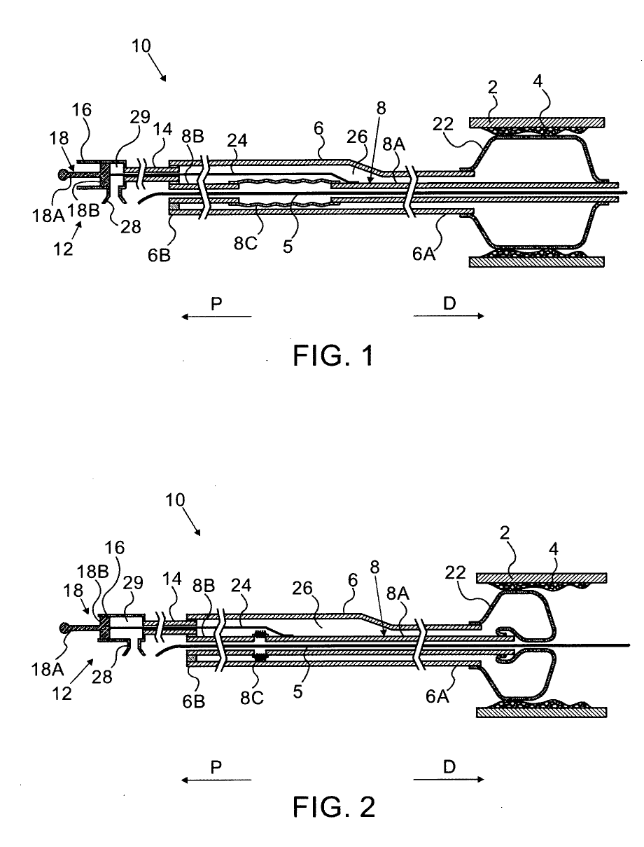

It is also noted that in the following description and in the claims of the present application, the terms “distal” and “proximal” are defined as follows: the catheter side or catheter end which is inserted into the body first is referred to as the distal side or distal end and the other (trailing) side or end of the catheters is referred to as the proximal side. For example, in the balloon catheter 10 of FIG. 1, the handle 12 is attached to the proximal end of the catheter 10 and the balloon 22 is disposed at the distal side of the catheter 10.

Similarly, when referring to sides, parts, ends or portions of any of the components, parts or portions of the catheters disclosed and illustrated in the present application, the term “distal” refers to a part, end or portion of the catheter component or part closer to the distal end of the catheter and the term “proximal” refers to the part, end or portion of the catheter component or part closer to the proximal end of the catheter. For example, in FIG. 1 the distal part 8A of the inner conduit 8 is closer to the distal end of the catheter 10 while the proximal part 8B of the inner conduit 8 is closer to the proximal end of the catheter 10. In another example, the end 6A of the outer conduit 6 is defined as the distal end of the outer conduit 6 and the end 6B of the outer conduit 6 is defined as the proximal end of the outer conduit 6.

Similarly, for moving parts of the catheters of the present application, moving proximally means moving in a direction generally defined by the arrow labeled P (in FIGS. 1-6) while moving distally means moving in a direction generally defined by the arrow labeled D (in FIGS. 1-6).

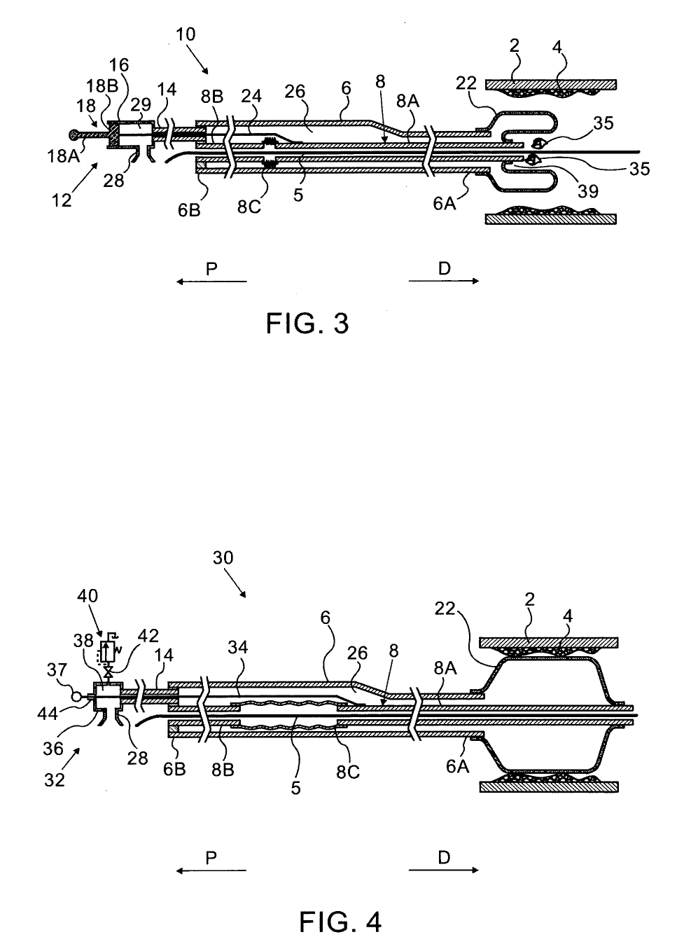

Reference is now made to FIGS. 1-3. FIG. 1 is a schematic cross-sectional diagram illustrating a rapid-exchange intususseptable balloon catheter having an inner conduit comprising a proximal part sealingly connected to a distal part by a variable length sleeve member, in accordance with an embodiment of the catheter of the present application. FIG. 2 is a schematic cross-sectional diagram illustrating the rapid exchange catheter of FIG. 1 with the balloon in an inflated and intussuscepted state achieved by proximally pulling a pulling member attached to the distal part of the inner conduit and causing the thin flexible part of the inner conduit to longitudinally shorten, in accordance with an embodiment of the catheter of the present application. FIG. 3 is a schematic cross-sectional diagram illustrating the rapid exchange catheter of FIGS. 1-2 with the balloon being in an intussuscepted and deflated state.

Turning to FIG. 1, the rapid-exchange balloon catheter 10 includes an outer conduit 6, an inner conduit 8 and an inflatable element such as the inflatable balloon 22. The outer conduit 6 is a hollow conduit and may be made from a suitable polymer based material such as but not limited to Nylon®, PEBAX® and the like, and may or may not be reinforced. The distal end 6A of the outer conduit 6 is sealingly attached to the proximal end of an inflatable balloon 22 by any suitable attachment method known in the art, such as, but not limited to, gluing by a suitable adhesive, or by ultrasonic welding attachment methods of by thermal bonding methods, or the like. The inner conduit 8 is a hollow conduit and is preferably (but not obligatorily) generally tubular in shape and includes a distal part 8A, a proximal part 8B and a sleeve member 8C. The distal part 8A and the proximal part 8B of the inner conduit 8 may be made from a suitable polymer based material such as, but not limited to a polyimide, Nylon®, reinforced polyimide, reinforced Nylon® and the like. Preferably, the material from which the distal part 8A and the proximal part 8B of the inner conduit 8 are made is a flexible material having a high resistance to kinking and a high resistance to longitudinal compression However, any other suitable material having the required mechanical characteristics may be used.

The variable length sleeve member 8C may be a thin walled flexible corrugated sleeve and may be made from a suitable polymer based material such as but not limited to PET (Polyethylene terephtalate), PTFE (polytetrafluorethylene), EPTFE (expandable polytetrafluorethylene), PEBAX®, Nylon® or any other material suitable for making a conduit that may suitably shorten in length or contract or shrink when the distal part 8A is pulled or moved proximally (such as, for example, when the plunger member 18 is pulled proximally).

It is noted that the sleeve member 8C may be also referred to as a contractible sleeve member or a shrinkable sleeve member to indicate that the sleeve member 8C (and 108C hereinafter) may vary in effective length by either contracting (or shrinking) or extending (lengthening) in the longitudinal (axial) direction of the catheter in response to a force applied thereto in the proximal direction or in the distal direction, respectively, the terms “variable length sleeve member”, “contractible sleeve member” and “shrinkable sleeve member” are interchangeably used hereinafter in referring to the sleeve member 8C (or to the sleeve member 108C of FIG. 6).

The distal part 8A of the inner conduit 8 is sealingly attached to the distal end of the inflatable balloon 22. The proximal end and the distal end of the balloon 22 may be sealingly attached to the distal part 8A and to the outer conduit 6, by any suitable attachment method known in the art, such as, but not limited to, gluing, welding, ultrasonic welding, thermal bonding and the like.

The proximal part 8B of the inner conduit 8 may be a straight part that is sealingly attached to and has an opening at the proximal end 6B of the outer conduit 6. A guide wire 5 may be inserted into the lumen of the inner conduit 8 and the catheter 10 may be moved along the guide wire 5 and inserted into a body lumen (such as, for example, a blood vessel) as is known in the art. The rapid- exchange catheter 10 also includes a handle assembly 12 and a connecting conduit 14 for connecting the handle assembly 12 to the outer conduit 6. The connecting conduit 14 is preferably a flexible hollow tubular member made from a stiff strong and flexible material such as, but not limited to, stainless steel in order to provide high pushability to the catheter as the connecting conduit 14 may be used to push the catheter to the target region. However, the connecting conduit 14 may also be made from other suitable materials having high pushability, such as for example, suitable reinforced polymer based materials including but not limited to, reinforced polyimide tubing or reinforced Nylon® tubing, and the like. The proximal end of the connecting conduit 14 is sealingly attached to the handle assembly 12 (by using any suitable attachment method known in the art, such as but not limited to a suitable glue or bonding compound or by thermal bonding and the like) and the distal end of the connecting conduit 14 is sealingly attached to the proximal end 6B of the outer conduit 6.

The handle assembly 12 includes a syringe-like member 16 having a plunger member 18 which is sealingly and movably disposed within the syringe-like member 16. The plunger member 18 includes a pulling rod 18A and a piston 18B attached to the pulling rod 18A. The piston 18B is sealingly and movably disposed within the syringe-like member 16 and may be longitudinally moved within the syringe-like member 16 in the proximal or distal directions by pulling or pushing the pulling rod 18A, respectively. The syringe-like member 16 includes an inflation port 28 formed therein. Alternatively, the inflation port 28 may be connected to the syringe-like member by an inflation tube (not-shown) as is known in the art. An indeflator device (not shown in FIGS. 1-6 for the sake of clarity of illustration) may be attached to the inflation port 28 for inflating the balloon 22 with an inflation fluid, through the lumen of the connecting conduit 14 and the space 26 formed between the outer conduit 6 and the inner conduit 8. The connecting conduit 14 is sealingly attached (by using any suitable attachment method known in the art, such as but not limited to a suitable glue or bonding compound or by thermal bonding and the like) to the syringe-like member 16 of the handle 12 such that the space 29 of the handle 12 is in fluidic communication with the space 26 of the catheter 30 through the lumen of the connecting conduit 14.

The rapid exchange catheter 10 also includes a pulling member 24. The pulling member 24 may be a thin strong, stiff and flexible wire made from a suitable metal such as but not limited to stainless steel (but other types of suitable materials, such as but not limited to, reinforced polymer based material, Kevlar®, and the like may also be used for implementing the pulling member 24). The first end of the pulling member 24 is attached to the distal end of the plunger member 18. The pulling member 24 passes through the inner space 29 of the syringe-like member 16 and through the space 26 formed between the outer conduit 6 and the inner conduit 8. The second end of the pulling member 24 is attached to the distal part 8A of the inner conduit 8.

In operation, the guide wire 5 may be inserted into a body lumen or suitable blood vessel (such as, but not limited to, using a femoral artery catheter insertion using an introducer sheath, as is well known in the art) and guided to a target region (such as, but not limited to, an obstructed coronary artery) as is known in the art. The rapid-exchange catheter 10 may then be inserted into the body by passing the catheter 10 over the guide wire 5 and pushing the catheter 10 until it reaches the intended target (such as, for example, a stenosed part of a coronary artery or other blood vessel). Alternatively, the rapid exchange catheter 10 may be inserted and guided to the target region by using a suitable type of guiding catheter, as is known in the art. After reaching the target region, the catheter 10 may then be positioned such that the non-inflated balloon 22 is positioned within the artery (or vein) region that is to be treated. An indeflator device (not shown) is then connected to the inflation port 28 and inflation fluid is inserted into the catheter 10 through the inflation port 28 to inflate the balloon 22.

FIG. 1 schematically illustrates the catheter 10 positioned within a blood vessel 2 such that the inflated balloon 22 is positioned within a region of the vessel 2 that is obstructed by plaque 4. The inflation of the balloon 22 by using a typical inflation pressure of 10-14 atmospheres may cause distending of the walls of the blood vessel 2 and compaction of the plaque 4 (However, it is noted that this inflation pressure range is given by way of example only and that other, different, inflation pressure values may be used, depending, inter alia, on the balloon type, balloon dimensions, balloon wall thickness, and other structural, mechanical and clinical considerations). After treatment of the stenosis, the pressure of the inflating fluid within the catheter 10 may be reduced by using the indeflator device while still keeping the balloon 22 in an inflated state. For example, the pressure of the inflation fluid may be reduced (by using the indeflator device as is known in the art) to 3 (three) atmospheres. However, other, different reduced pressure values, higher or lower than three atmospheres, may also be used depending inter alia on the characteristics of the balloon 22, the particular clinical application and other considerations.

After the pressure reduction, the physician or the operator of the catheter 10 pulls the plunger 18 in the proximal direction. As the plunger 18 is pulled proximally, the distal part 8A of the inner conduit 8 is pulled proximally by the pulling member 24. The pulling force acting on the part 8A causes the thin walled flexible sleeve member 8C to collapse under the force acting on it and to shorten in the longitudinal direction such that the distance between the part 8A and the part 8B is reduced as the part 8B moves proximally towards the part 8B. As the part 8A moves proximally within the outer conduit 6, the distal end of the inflated balloon 22 collapses and the balloon 22 intussuscepts by folding inwardly (invaginating) as illustrated in FIG. 2. It is noted that, preferably (but not obligatorily), the extended (non-shortened) length of the sleeve member 8C is larger than the length of the balloon 22 to allow full intussuscepting of the balloon 22 when the sleeve member 8C shortens as the distal part 8B is proximally pulled by the pulling member 24. Such full intussuscepting results in the length of the fully intussuscepted balloon 22 being about half (or slightly less than half) of the length of the non-intussuscepted balloon 22.

It is noted however, that while preferably, the catheters of the present application are constructed to enable full intussuscepting of the balloon 22 in order to maximize the length and volume of the cavity 39 (best seen in FIG. 3) so as to increase the volume available for trapping debris or particulate matter, this is not obligatory and in accordance with other embodiments of the invention, the length of the fully extended sleeve member may be equal to or shorter than half the length of a non-intussuscepted balloon. In such embodiments, the balloon 22 may not be fully intussuscepted after maximal shortening of the sleeve like member is achieved and the length of the balloon after maximal shortening of the sleeve like member in larger than half of the length of the fully extended balloon 22. This embodiment may be implemented in catheters having particularly long balloons, or in applications in which it may not be desired to increase the length of the fully extended sleeve member beyond a length due to mechanical and/or fluidic considerations.

Thus, in accordance with three different embodiments of the catheters of the present application, the length of the variable length sleeve member 8C in the fully extended state may be either smaller than half the length of the balloon when said balloon is fully extended, or equal to half the length of said balloon when said balloon is fully extended, or larger than half the length of said balloon when said balloon is fully extended.

As the intussuscepting of the balloon 22 shortens the balloon length and reduces the volume inside the intussuscepted balloon, the inflation fluid that is ejected from within the balloon 22 during the intussuscepting passes into the space 29. The volume of the space 29 gradually increases due to the proximal movement of the piston 18B of the plunger 18, thereby accommodating at least some of the inflation fluid being ejected from the balloon 22.

It is noted that, in accordance with an embodiment of the catheters of the present application, the dimensions of the syringe-like member 16 and of the piston 18A may be prearranged such that the space 29 within the syringe-like member 16 fully accommodates the volume of inflation fluid ejected from the balloon 22 during the intussuscepting of the balloon 22 and prevents a substantial increase in the pressure within the catheter 10 and the balloon 22 during the intussuscepting of the balloon 22. However, while being preferred, this arrangement is not obligatory and the dimensions of the syringe-like member 16 may be such that not all of the volume ejected from the balloon 22 is accommodated within the space 29 formed within the syringe-like member 16 due to the proximal movement of the piston 18B. In such cases one or more other pressure regulating or pressure controlling mechanisms may be used to avoid uncontrolled pressure increase within the balloon 22 during the proximal movement of the plunger 18B. Such pressure regulating mechanisms or pressure controlling mechanisms (including, but not limited to, pressure relief valves, hydraulic accumulators, and the like) are described in detail hereinafter and illustrated in FIGS. 4-6.

It is further noted that in accordance with an embodiment of the catheters of the present more than one type of pressure controlling mechanism may be use in the catheters disclosed in the present application. Thus, any suitable pressure controlling mechanism(s) may be used in the catheters of the present application. Such pressure controlling mechanism combinations may be selected from, inter alia, a syringe-like member, a pressure relief valve and a hydraulic capacitor and any combinations thereof.

After the intussuscepting of the balloon 22 is completed, the balloon 22 is deflated. The deflating of the balloon 22 may be performed by using the indeflator to apply negative pressure to the catheter 10, as is known in the art. As the pressure within the balloon 22 is reduced, the balloon 22 shrinks and a cavity 39 is formed in the invaginated part of the balloon 22. As the formed cavity 39 is open to the lumen of the blood vessel 2, the suction created by the expansion of the cavity 39 during deflation of the balloon 22 causes blood and/or plaque particles and/or debris 35 and/or other particulate matter released during treatment of the obstruction to be withdrawn into the cavity 39 of the intussuscepted balloon 22.

After the deflating of the balloon 22, the rapid-exchange catheter 10 may be withdrawn proximally and taken out of the body together with any debris 35 and/or particulate matter entrapped in the cavity 39 of the balloon 22.

Once the catheter 10 is withdrawn outside the body, the material trapped within the intussuscepted balloon 22 may be collected by pushing the plunger 18 distally to move the part 8A of the inner conduit 8 distally thereby everting (turning inside-out) the invaginated balloon 22 into a non-invaginated shape while enabling the collecting samples of trapped plaque debris 35 or other particulate matter or other biomaterial which was collected in the space 39 of the balloon 22. Such collected material may be subjected to further analysis (chemical, physical, pathological or other types of analysis may be performed on the collected, as is known in the art) if desired.

It is noted that when the catheter 10 is placed in a blood vessel, the target site may be in the vicinity of a stenosed portion of the blood vessel. In such a case the method may include a step of introducing a medical device through the lumen of the inner conduit 8 for treating a stenosed portion in the blood vessel 2 (more than one stenosed portion may be treated by such a medical device). Such a medical device (or medical devices may be a diagnostic device for diagnosing one or more portions of the blood vessel 2, or a treatment device for treating one or more portions of the blood vessel 2, or a combined treatment device and diagnostic device for performing a diagnostic procedure before during or after a treatment procedure performed on the blood vessel 2.

It is noted that the term “diagnostic device' as used throughout the present application also includes sensing devices which do not produce a diagnosis but which provide data which may be further processed to obtain data of diagnostic value. Thus the term “diagnostic device” as used throughout the present application, defines any device which includes one or more sensors capable of sensing any type of biologically relevant physical and/or chemical and/or mechanical and/or electrical and/or electromagnetic parameters within the body passage or blood vessel and of producing signals representing the sensed parameters. The values of the parameters/signals sensed by such sensor based devices may be sent out of the body for further processing to obtain diagnostic data or, alternatively, may be processed into data by the medical device and the resulting data may be sent out as is known in the art. The sending of such signals or processed data may be performed by wire or may be wirelessly transmitted out of the device using a transmitter and or transceiver to communicate with an external receiver or transceiver disposed outside the body as is well known in the art of telemetry.

Such diagnostic devices and/or sensing devices may be passive devices (for passively sensing the value of the measured parameter in the target region), such as but not limited to, sensing electrodes for sensing electrical signals, temperature sensors for sensing the temperature, chemical sensors for sensing the concentration of a chemical species and the like, or may be active (actively sending out signals and collecting returning or modified signals), such as but not limited to ultrasonic transducers, MRI probes, spectrometer devices, and the like.

In accordance with the methods disclosed herein, the medical treatment device may include but is not limited to, a mechanical atherectomy device, a laser atherectomy device, an ultrasonic treatment device, an embolic protection device, a filter device, a basket-like device, a blade-like device, an occluding balloon device, an aspirating device and any combinations thereof. However, any other medical treatment device known in the art which may be inserted through the lumen of the inner conduit 8 (or 108) of the catheters of the present application may be inserted into the catheter and used for performing treatment of the blood vessel 2 as is well known in the art of rapid exchange catheters.

In accordance with other embodiments of the methods disclosed herein, the medical diagnostic device may include but is not limited to, an imaging device, an infra-red imaging device, an imaging device operating in the visual range of the electromagnetic radiation spectrum, an ultrasonic imaging device, a magnetic resonance imaging device, an electrode based sensing device, a temperature sensing device, an electrochemical sensing device, a device for sensing the concentration of a chemical species and any combinations thereof.

In accordance with other embodiment of the method disclosed herein, the medical device may be used for treating said blood vessel (or other body passage) in which the catheter is disposed. Typically, such treating steps may occur after the balloon 22 is inflated in the vicinity of a stenosed or occluded region of a blood vessel for example, the distal end of the catheter 10 may be placed near such a stenosed blood vessel region, the balloon 22 may then be inflated to anchor the catheter in place and an athetectomy device catheter may then be inserted through the lumen of the inner conduit and advanced to the stenosed region to treat the stenosed region and open the stenosis, as is well known in the art. Preferably, after treatment is completed, the method may include withdrawing said treating device outside of the catheter (and/or outside the body) by pulling the treating device proximally through the lumen of the inner conduit 8 prior to intussuscepting the balloon 22 (by pulling the pulling rod 18 proximally). Alternatively, the step of intussuscepting of the balloon 22 and/or the step of deflating the balloon may be performed prior to withdrawing of the treating medical device, and the treating device may be withdrawn from the body together with the catheter at the end of the treatment procedure or may be withdrawn after deflating the balloon 22.

Similarly, in accordance with an embodiment of the method disclosed herein, in cases where the medical device includes a diagnostic device or is a diagnostic device, the method may include the step of performing a diagnostic procedure on at least a portion of said blood vessel using the diagnostic medical device. The diagnostic procedure may be performed at any stage after the inserting of the diagnostic medical.

In accordance with an embodiment of the method disclosed herein, the method may include a step of introducing a stent (not shown) into the body passage or blood vessel using the balloon and a step of deploying the stent within the body passage or blood vessel during the step inflating of the balloon 22. The stent may be disposed (in a non-expanded state) on the balloon 22 prior to inserting the catheter 10 into the body, as is well known in the art. After insertion of the balloon 22 carrying the stent into the stenosed target region or a portion of a stenosed target region, the stent may be deployed in the target region by the inflating of the balloon 22 as disclosed herein. Methods of stent deployment using a balloon catheter are well known in the art and are therefore not described in detail hereinafter. The stent may be any type of stent known in the art which is suitable for insertion into a body and deployment in a body passage or blood vessel using a balloon catheter. After the stent is deployed in its expanded state, the operator of the catheter 10 may proceed by intussuscepting the balloon 22 as described in detail and deflating the balloon to trap debris and/or any particulate matter released by the introduction and/or deployment of the stent. If the stenosed region was treated by a medical treatment device as disclosed hereinabove (such as, for example, by an atherectomy device for opening a stenosed region), the stent positioning and deployment may be performed after treating the stenosed region.

It is noted that when a diagnostic medical device is being used together with stent deployment by the balloon 22 and /or together with treating of the body passage or blood vessel with a treating device, the diagnostic procedure may be performed before, and/or after the treatment of the body passage or blood vessel to obtain data representing the state of the blood vessel or passage before and after the treatment. Similarly, the diagnostic procedure may be performed before, and/or after stent deployment. The position of the diagnostic medical device may be changed by suitably moving (pushing or pulling the diagnostic device to position the device in the desired target region. After the deflating of the balloon 22 is performed to trap the debris 25 it is still possible to change the position of the diagnostic device by either pushing or pulling it within the lumen of the inner conduit 8 of the catheter 10 or by moving the entire catheter 10 proximally or distally within the blood vessel (or body passage) together with the diagnostic device. In this way it is possible to obtain data representative of the state of desired (accessible) portions of the blood vessel or body passage vessel before the balloon 22 is inflated and/or after the balloon 22 is deflated.

In accordance with one non-limiting example of a specific embodiment of the catheter 10, the outer conduit 6 may be a cylindrical tube having an inner diameter of 0.7 millimeter an outer diameter of 0.9 millimeter and a wall thickness of 0.1 millimeter. The parts 8A and 8B of the inner conduit 8 may be cylindrical tubes having an outer diameter of 0.5 millimeter, and inner diameter of 0.4 millimeter and a wall thickness of 0.05 millimeter. The variable length sleeve member 8C may be a cylindrical (or corrugated) sleeve having an inner diameter of 0.5 millimeter, an outer diameter of 0.55 millimeter and a wall thickness of 0.025 millimeter, the length of the sleeve member 8C may be 25 millimeter. The length of the balloon 22 may be 20 millimeter and the inflated outer diameter of the balloon 22 may be 3.0 millimeter. The pulling member 24 may be a wire made from SS 304L stainless steel and having a diameter of 0.05 millimeter.

It is noted that the dimensions specified above are given by way of example only, are not obligatory and are not intended to be limiting. Rather, any of the dimensions specified hereinabove may be modified and changed (increased or decreased) and other different dimensions may be used in implementing the catheters of the present application depending, inter alia on the specific application, the particular type of the catheter being used, the characteristics of the materials used in the various catheter components and on other design, manufacturing and operational considerations.

It is noted that while the sleeve member 8C is implemented as a sleeve having a concertina-like or accordion-like or corrugated shape (as illustrated in FIGS. 1-3) to assist the longitudinal shortening of the flexible sleeve 8C when the plunger 18 is pulled proximally, this is not obligatory and the sleeve member 8C may also be implemented as a thin walled flexible non-corrugated cylindrical sleeve, as long as the sleeve is capable of shortening along it's longitudinal axis when the distal end 8A of the inner conduit 8 is pulled proximally by the pulling member 24. Such shortening may occur by crimping or crumpling or any type of irregular folding of such a (cylindrical or non-cylindrical) sleeve member to effectively shorten the sleeve member in the longitudinal direction and to shorten the distance between the parts 8A and 8B of the inner conduit 8, resulting in intussuscepting of the balloon 22.

Furthermore, in accordance with other embodiments of the catheters of the present application, the sleeve like member may be selected from a cylindrical sleeve like member having a circular cross section, a sleeve like member having a non-circular cross section (such as an elliptical cross section or any other different cross section shape) and a sleeve like member having at least one corrugated portion and at least one non-corrugated portion. For example, the distal end and the proximal end of the sleeve like member may have a non-corrugated circular cross section to facilitate attachment of the sleeve member's ends to the proximal part of the inner conduit and to the distal part of the inner conduit while the middle portion of the sleeve portion may be corrugated as shown in FIG. 1. In accordance with another embodiment, the sleeve member may have two or more corrugated portions separated by non-corrugated sleeve portions.

It is noted that while the handle 12 of FIG. 1-3 is used for intussuscepting the balloon 22 and for simultaneously accommodating excess of inflation fluid ejected from the balloon 22 during the intussuscepting to avoid substantial pressure increase within the catheter 10, it is possible to modify the catheter 10 by using other different handle configurations including one or more pressure controlling mechanisms for allowing intussuscepting the balloon 22 without causing a substantial pressure increase in the balloon 22.

Reference is now made FIG. 4 which is a schematic cross-sectional diagram illustrating a rapid-exchange intussuscepting balloon catheter having a handle including a pressure relief valve, in accordance with an embodiment of the catheter of the present application.

The catheter 30 is similar in structure to the catheter 10 of FIGS. 1-3 except that the catheter 30 include a handle 32 instead of the handle 12 of catheter 10 and the pulling member 34 of the catheter 30 is different than the pulling member 24 of the catheter 10.

The outer conduit 6, the inner conduit 8, the connecting conduit 14 and the balloon 22 of the catheter 30 are identical to the outer conduit 6, the inner conduit 8, the connecting conduit 14 and the balloon 22 of the catheter 30.

The handle 32 of the catheter 30 includes a hollow housing 36 having a space 38 therein. The housing 36 includes an inflation port 28 for fluidically connecting a standard indeflator device (not shown, for the sake of clarity of illustration) to the space 38. The indeflator device may provide inflation fluid through the space 38, the connecting conduit 14 and the space 26 for inflating the balloon 22. The connecting conduit 14 is attached to the housing 36 of the handle 32 such that the space 38 is in fluidic communication with the space 26 of the catheter 30 through the lumen of the connecting conduit 14. The handle 32 also includes a pressure relief valve 40 and a closable stopcock 42. The relief valve 40 is fluidically connected to the space 38 through the closable stopcock 42 such that when the closable stopcock 42 is closed, the relief valve is fluidically isolated from the space 38 and when the closable stopcock 42 is opened, the relief valve 40 is fluidically in communication with the space 38 and with the internal space of the catheter 30 and the internal space within the balloon 22. The relief valve 40 may be an overpressure valve which is configured such that when the pressure within the space 38 exceeds a threshold value (an exemplary value of such a pressure threshold value may be, but is not limited to, 3.5 atmospheres), the relief valve 40 opens and discharges an amount of inflation fluid until the pressure within the space 38 is reduced to be equal to or below the preset pressure threshold and the relief valve 40 closes.

Preferably, the pressure threshold value of the pressure relief valve is preset at the factory at a value which depends, inter alia, on the size and other physical parameters of the balloon 22 and on the particular application (such as, but not limited to angioplasty, angioplasty with stent deployment, atherectomy and angioplasty, atherectomy and angioplasty with Stent deployment, and the like).

The pulling member 34 may be a thin strong and flexible wire made from a suitable metal such as but not limited to stainless steel (but other types of different suitable materials, such as Kevlar® may also be used for implementing the pulling member 34).

A holding member 37 may be attached to the first end of the pulling member 34. The holding member 37 may be formed as a knob having a size and shape which may be conveniently held and manipulated by an operator using the catheter. The holding member 37 may be used for pulling the pulling member 34 proximally (and for pushing the pulling member distally, if necessary). The pulling member 34 passes into the space 38 through a sealed entry port 44 in the housing 36 and extends through the lumen of the connecting conduit 14 and through the space 26 formed between the outer conduit 6 and the inner conduit 8. The second end of the pulling member 34 is attached to the distal part 8A of the inner conduit 8. When the holding member 37 is pulled in the proximal direction, the distal part 8A of the inner conduit 8 moves proximally to shorten the flexible sleeve in the longitudinal direction and to cause intussuscepting of the balloon 22 (as disclosed in detail hereinabove for the catheter 10).

In operation, the catheter 30 may be inserted into the body by using a guide wire 5 as disclosed in detail hereinabove with respect to the catheter 10 until the balloon 22 is placed in the obstructed region of the blood vessel to be treated. An indeflator is sealingly attached to the catheter 30 at the inflation port 28 and the closable stopcock 42 is closed.

FIG. 4 schematically illustrates the catheter 30 positioned within a blood vessel 2 such that the inflated balloon 22 is positioned within a region of the vessel 2 that is obstructed by plaque 4. The indeflator may then be used to inflate of the balloon 22 by using a typical inflation pressure of 10-14. The inflated balloon 22 may cause distending of the walls of the blood vessel 2 and compaction of the plaque 4 (However, it is noted that this inflation pressure range is given by way of example only and that other, different, inflation pressure values may be used, depending, inter alia, on the balloon type, balloon dimensions, balloon wall thickness, and other structural, mechanical and clinical considerations). After treatment of the stenosis, the pressure of the inflating fluid within the catheter 30 may be reduced while still keeping the balloon 22 in an inflated state. For example, the pressure of the inflation fluid may be reduced to 3 atmospheres (However, it is noted that other, different reduced pressure values may also be used at this stage).

After the pressure reduction, the physician (or the operator) of the catheter 30 opens the closable stopcock 42. After opening of the closable stopcock 42, the physician pulls the holding member 37 in the proximal direction. As the holding member 37 is pulled proximally, the distal part 8A of the inner conduit 8 is pulled proximally by the pulling member 34. The pulling force acting on the part 8A causes the sleeve member 8C to collapse under the force acting on it and to shorten in the longitudinal direction such that the distance between the part 8A and the part 8B is reduced as the part 8B moves proximally towards the part 8B. As the part 8A moves proximally within the outer conduit 6, the distal end of the inflated balloon 22 collapses and the balloon 22 intussuscepts by folding inwardly (invaginating) as disclosed hereinabove with respect to the balloon 22 of catheter 10 (see FIG. 2).

As the intussuscepting of the balloon 22 shortens the balloon length, the pressure inside the catheter 30 increases. When the pressure inside the catheter 30 exceeds the preset pressure threshold of the relief valve 40, the relief valve 40 opens and discharges inflation fluid. For example, if the preset pressure threshold value of the relief valve 40 is 3.5 atmospheres, the relief valve 40 will open and discharge inflation fluid each time the pressure in the space 38 exceeds 3.5 atmospheres. The relief valve 40 enables inflation fluid that is ejected from within the balloon 22 during the intussuscepting of the balloon 22 to pass into the space 38 and to exit the catheter 30 by being discharged from the relief valve 40. The discharging of inflation fluid through the relief valve 40 allows the intussuscepting of the balloon 22 to proceed and prevents the internal pressure within the catheter 30 from exceeding the pressure threshold of the relief valve 40 (which is 3.5 atmospheres for the non-limiting example disclosed hereinabove).

After the intussuscepting of the balloon 22 is completed, the balloon 22 is deflated using the indeflator device (not shown), as disclosed hereinabove for catheter 10. As the pressure within the balloon 22 is reduced, the balloon 22 shrinks and a space is formed in the invaginated part of the balloon 22 (as shown in detail for the balloon 22 of catheter 10 and illustrated in FIG. 2). Plaque debris and particulate matter may then be captured by the intussuscepted balloon 22 as disclosed in detail hereinabove for the balloon 22 of the catheter 10. After the deflating of the balloon 22, the rapid-exchange catheter 30 may be withdrawn proximally and taken out of the body together with any debris and/or particulate matter entrapped in the balloon 22. Once the catheter 30 is withdrawn outside the body, the material trapped within the intussuscepted balloon 22 may be collected and subjected to further analysis as described in detail hereinabove.

The use of a pressure relief valve 42 has certain advantages. For example, the same relief valve may be used in catheters having different balloon lengths and/or balloon diameters since in a catheter having a relief valve there is no need to accommodate the different volumes of inflation fluid ejected during the intussuscepting of different balloons having different balloon lengths and/or different balloon diameters because the inflation fluid ejected from such different balloons is discharged outside the catheter and need not be accommodated within a part of the catheter as is the case for catheter 10 in which the amount of ejecting fluid ejected from the balloon has to be accommodated by the syringe-like member 16.

However, it should be born in mind that it is possible to use alternative catheter designs to deal with the pressure increase and excess fluid accommodation during intussuscepting the balloon of the catheter.

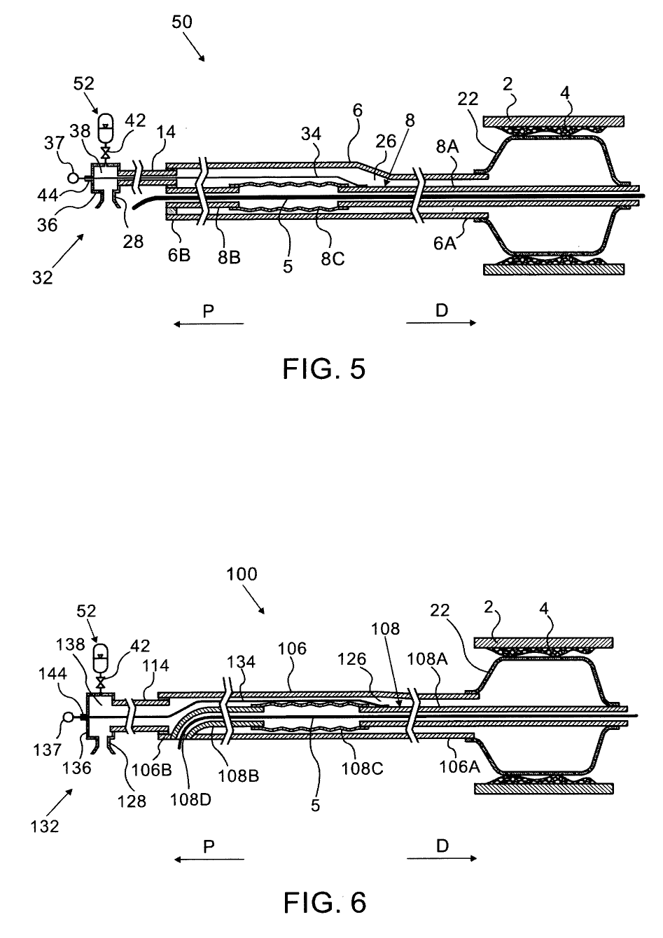

Reference is now made to FIG. 5 which is a schematic cross-sectional diagram illustrating a rapid-exchange intussusceptable balloon catheter having a hydraulic accumulator, in accordance with an embodiment of the catheter of the present application.

The catheter 50 is similar to the catheter 30 except that in the catheter 50 includes a hydraulic accumulator 52, instead of the relieve valve 40 (of catheter 30). The hydraulic accumulator 52 of the catheter 50 is connected to a closable stopcock 42. The stopcock 42 may be closed to fluidically isolate the hydraulic accumulator 52 from the space 38. The stopcock 42 may also be opened to fluidically connect the hydraulic accumulator 52 to the space 38.

It is noted that the stopcock 42 is optional and is not obligatory to the operation of the catheter 50. Therefore, in accordance with an alternative embodiment of the catheter 50, the catheter 50 does not include the stopcock 42 and the hydraulic accumulator 52 is directly fluidically connected to the space 38 of the handle 32.

The hydraulic accumulator 52 is designed to accommodate fluid ejected from the balloon 22 during intussuscepting thereof. The structure and operating of hydraulic accumulators is well known in the art, is not the subject of the present application and is therefore not described in detail in the present application.

Briefly, a hydraulic accumulator is designed to accommodate excess fluid while preventing excessive increase in the pressure in a fluidic system to which it is fluidically connected. This may be achieved by several different designs such as but not limited to hydraulic accumulators using a bladder, hydraulic accumulators using a moving piston disposed in a compressible gas chamber, hydraulic accumulators using a chamber with a spring loaded piston therein, and other types of hydraulic accumulators as is well known in the art. It is noted that in FIG. 5, the hydraulic accumulator 52 is represented by the conventional engineering symbol labeled 52 and is not drawn to scale).

When fluid ejected from the balloon 22 of the catheter 50 into the hydraulic accumulator 52, the pressure increases somewhat, but as the volume available within the hydraulic accumulator 52 is relatively large in comparison with the volume of fluid ejected from the balloon 22 during intussuscepting thereof the pressure increase within the catheter 50 is attenuated and is not large enough to prevent the intussuscepting of the balloon 22. The dimensions, accommodated volume and other characteristics of the hydraulic accumulator 52, such as the maximal pressure developed in the catheter after the balloon 22 has been fully intussuscepted may be selected depending, inter alia, on the dimensions of the balloon 22, the volume ejected from the balloon 22 during intussuscepting, the balloon's inflation pressure, and other design considerations.

In operation, the catheter 50 may be inserted into the body by using a guide wire 5 as disclosed in detail hereinabove with respect to the catheter 10 until the balloon 22 is placed in the obstructed region of the blood vessel to be treated. An indeflator (not shown for the sake of clarity of illustration) is sealingly attached to the catheter 50 at the inflation port 28 and the closable stopcock 42 is closed in order to fluidically isolate the hydraulic accumulator 52 from the catheter 50 and to prevent any movement of inflation fluid into the hydraulic accumulator 52 during the initial balloon inflation step.

FIG. 5 schematically illustrates the catheter 50 positioned within a blood vessel 2 such that the inflated balloon 22 is positioned within a region of the vessel 2 that is obstructed by plaque 4. The indeflator (not shown) may then be used to inflate the balloon 22 by using a typical inflation pressure of 10-14 atmospheres. The inflated balloon 22 may cause distending of the walls of the blood vessel 2 and compaction of the plaque 4 (However, it is noted that this initial inflation pressure range is given by way of example only and that other, different, inflation pressure values may be used, depending, inter alia, on the balloon type, balloon dimensions, balloon wall thickness, and other structural, mechanical and clinical considerations). After treatment of the stenosis, the pressure of the inflating fluid within the catheter 50 may be reduced while still keeping the balloon 22 in an inflated state. For example, the pressure of the inflation fluid may be reduced to 3 atmospheres or slightly higher than 3 atmospheres (However, it is noted that other, different reduced pressure values may also be used at this reduced pressure inflation stage).

After the pressure reduction, the physician (or the operator) of the catheter 50 opens the closable stopcock 42 to fluidically connect the hydraulic accumulator 52 to the space 38. As the connecting of the space to the hydraulic accumulator 52 may result in a certain reduction in the pressure due to movement of some inflation fluid into the hydraulic accumulator 52, it may be necessary to increase the pressure again to 3 atmospheres by using the indeflator which is attached to the inflation port 28 to increase the pressure to the desired value of about 3 atmospheres. After opening of the closable stopcock 42, the physician pulls the holding member 37 in the proximal direction. As the holding member 37 is pulled proximally, the distal part 8A of the inner conduit 8 is pulled proximally by the pulling member 34. The pulling force acting on the part 8A causes the sleeve member 8C to collapse under the force acting on it and to shorten in the longitudinal direction such that the distance between the part 8A and the part 8B is reduced as the part 8B moves proximally towards the part 8B. As the part 8A moves proximally within the outer conduit 6, the distal end of the inflated balloon 22 collapses and the balloon 22 intussuscepts by folding inwardly (invaginating) as disclosed hereinabove with respect to the balloon 22 of catheter 10 (see FIG. 2).

As the intussuscepting of the balloon 22 shortens the balloon length, excess inflation fluid is ejected from the balloon 22 causing a volume of inflation fluid to enter the hydraulic accumulator 52 and enabling full intussuscepting of the balloon 22 with a relatively moderate pressure increase within the fluidic system due to the buffering (attenuating) effect of the hydraulic accumulator. After the intussuscepting of the balloon 22 is completed, the pressure level in the balloon 22, in the relevant passages of the catheter 50 and within the hydraulic accumulator 52 is somewhat higher than the initial pressure of 3 atmospheres in the balloon 22 before starting the intussuscepting of the balloon 22. This precise pressure level is determined, inter alia, by the volume of inflation fluid ejected from the balloon 22, the volume and other characteristics of the hydraulic accumulator 52 and other characteristics of the catheter 50.

After the intussuscepting of the balloon 22 is completed, the balloon 22 is deflated by using the indeflator as described hereinabove. As the pressure within the balloon 22 is reduced, the balloon 22 shrinks and a cavity 39 is formed in the invaginated part of the balloon 22 (as shown in detail for the balloon 22 of catheter 10 and illustrated in FIG. 2). Plaque debris and particulate matter may then be drawn into and captured within the space formed within the intussuscepted balloon 22 as disclosed in detail hereinabove for the balloon 22 of the catheter 10. After the deflating of the balloon 22, the rapid-exchange catheter 50 may be withdrawn proximally and taken out of the body together with any debris and/or particulate matter entrapped in the balloon 22. Once the catheter 50 is withdrawn outside the body, the material trapped within the intussuscepted balloon 22 may be collected and subjected to further analysis as described in detail hereinabove.

It is noted that the method of operating the catheter 50 disclosed hereinabove is not obligatory and that other different methods may be used. For example, the alternative embodiment of the catheter 50 (which does not include the stopcock 42) may be operated by using a different method. The catheter 50 may be positioned within a blood vessel 2 such that the inflated balloon 22 is positioned within a region of the vessel 2 that is obstructed by plaque 4 (as illustrated in FIG. 5). An indeflator (not shown) may then be fluidically connected to the catheter 50 through the inflation port 28. The indeflator may then be used to inflate of the balloon 22 by using a typical inflation pressure of 10-14 atmospheres. It is noted that since in this method the hydraulic accumulator 52 is fluidically coupled to the space 38, during the inflation of the balloon 22 at a pressure of 10-14 atmospheres, some inflation fluid also flows into the hydraulic accumulator 52. The inflated balloon 22 may cause distending of the walls of the blood vessel 2 and compaction of the plaque 4 (However, it is noted that this initial inflation pressure range is given by way of example only and that other, different, inflation pressure values may be used, depending, inter alia, on the balloon type, balloon dimensions, balloon wall thickness, and other structural, mechanical and clinical considerations). After treatment of the stenosis, the pressure of the inflating fluid within the catheter 50 may be reduced while still keeping the balloon 22 in an inflated state. For example, the pressure of the inflation fluid may be reduced to 3 atmospheres or slightly higher than 3 atmospheres (However, it is noted that other, different reduced pressure values may also be used at this reduced pressure inflation stage).

After the pressure reduction, the physician or the operator of the catheter 50 pulls the holding member 37 in the proximal direction. As the holding member 37 is pulled proximally, the distal part 8A of the inner conduit 8 is pulled proximally by the pulling member 34. The pulling force acting on the part 8A causes the thin walled flexible sleeve 8C to collapse under the force acting on it and to shorten in the longitudinal direction such that the distance between the part 8A and the part 8B is reduced as the part 8B moves proximally towards the part 8B. As the part 8A moves proximally within the outer conduit 6, the distal end of the inflated balloon 22 collapses and the balloon 22 intussuscepts by folding inwardly (invaginating) as disclosed hereinabove with respect to the balloon 22 of catheter 10 (see FIG. 2).

As the intussuscepting of the balloon 22 shortens the balloon length, excess inflation fluid is ejected from the balloon 22 causing a volume of inflation fluid to enter the hydraulic accumulator 52 and enabling full intussuscepting of the balloon 22 with a relatively moderate pressure increase within the fluidic system due to the buffering (attenuating) effect of the hydraulic accumulator 52. After the intussuscepting of the balloon 22 is completed, the pressure level in the balloon 22, in the relevant passages of the catheter 50 and within the hydraulic accumulator 52 is somewhat higher than the initial pressure of 3 atmospheres in the balloon 22 before starting the intussuscepting of the balloon 22. This precise pressure level is determined, inter alia, by the volume of inflation fluid ejected from the balloon 22, the volume and other characteristics of the hydraulic accumulator 52 and other characteristics of the catheter 50.

After the intussuscepting of the balloon 22 is completed, the balloon 22 may be deflated by using the indeflator as described hereinabove. As the pressure within the balloon 22 is reduced, the balloon 22 shrinks and a space is formed in the invaginated part of the balloon 22 (as shown in detail for the balloon 22 of catheter 10 and illustrated in FIG. 2). Plaque debris and other particulate matter may then enter and become trapped in the space formed in the intussuscepted balloon 22 as described hereinabove with respect to catheter 10 and the catheter 50 together with any entrapped debris and/or particulate matter may be withdrawn from the body as disclosed in detail hereinabove with respect to the catheter 10.

The use of the hydraulic accumulator 52 has certain advantages. For example, as the fluidic system comprising the indeflator, the catheter 50 and the hydraulic accumulator 52 is a closed system, as long as the indeflator is attached to the inflation port 28, the inflation fluid ejected from the balloon 22 during intussuscepting of the balloon 22 is fully accommodated by the hydraulic accumulator 52 and is not ejected out of the system (as occurs with the system 30 using a relief valve), resulting in a cleaner operation of the device.

However, it is noted that catheters using different balloons with different internal volumes may require the use of different hydraulic accumulators with differing volume and pressure buffering capacities, depending, inter alia, on the amount of inflation fluid ejected from the different balloon sizes during intussuscepting of the balloon and on the maximal level of pressure allowable within the balloon 22 during the intussuscepting thereof. Thus, the type and characteristics of the hydraulic accumulator 52 may need to be adapted to one or more of the characteristics of the balloon 22, such as, but not limited to, the volume of the inflated balloon 22, the wall thickness of the balloon 22, the diameter of the balloon 22, the length of the balloon 22 and the like.

It is noted that while in the embodiments of the rapid exchange catheters 10, 30 and 50 disclosed hereinabove and illustrated in FIGS. 1-3, 4 and 5, respectively, the proximal end of the proximal part 8B of the inner conduit 8 is a straight tubular conduit (as illustrated in FIGS. 1-5), this is not obligatory and at least the proximal part of the proximal part of the inner conduit may be curved of the proximal part 8B to be attached to the side of the outer conduit (See FIG. 6) such that the proximal end of the lumen of the proximal part of the inner conduit opens on the side of the outer conduit of the catheter.

Reference is now made to FIG. 6 which is a schematic cross-sectional diagram illustrating a rapid exchange intussuscepting balloon catheter including a curved inner conduit having a lumen that opens on the lateral side of the outer conduit of the catheter, in accordance with another embodiment of the catheters of the present application.

The rapid-exchange balloon catheter 100 includes an outer conduit 106, an inner conduit 108 having a curved part 108B and an inflatable element such as the inflatable balloon 22. The outer conduit 106 is a hollow conduit and may be made from a suitable polymer based material such as but not limited to Nylon®, PEBAX® and the like, and may or may not be reinforced. The distal end 106A of the outer conduit 106 is sealingly attached to the proximal end of an inflatable balloon 22 by any suitable attachment method known in the art, such as, but not limited to, gluing by a suitable adhesive, or by ultrasonic welding attachment methods of by thermal bonding methods, or the like. The inner conduit 108 is a hollow conduit and is preferably tubular and includes a straight distal part 108A, a partially curved proximal part 108B and a sleeve member 108C. The distal part 108A and the proximal part 108B of the inner conduit 108 may be made from a suitable polymer based material such as, but not limited to a polyimide, Nylon®, reinforced polyimide, reinforced Nylon® and the like. Preferably, the material from which the distal part 108A and the proximal part 108B of the inner conduit 108 are made is a flexible material having a high resistance to kinking and a high resistance to longitudinal compression However, any other suitable material having the required mechanical characteristics may be used.

The sleeve member 108C may be a thin walled flexible corrugated sleeve and may be made from a suitable polymer based material such as but not limited to PET (Polyethylene terephtalate), PTFE (polytetrafluorethylene), EPTFE (expandable polytetrafluorethylene), PEBAX®, Nylon®, and the like. The distal part 108A of the inner conduit 108 is sealingly attached to the distal end of the inflatable balloon 22. The distal end of the balloon 22 may be attached to the distal end of the distal part 108A by any suitable attachment method known in the art, such as, but not limited to, gluing, welding, ultrasonic welding, thermal bonding and the like.

The proximal part 108B of the inner conduit 108 is curved laterally such that it is sealingly attached to and has an opening at the lateral side of the proximal end 106B of the outer conduit 106 such that a guide wire 5 may be inserted into the open lumen of the inner conduit 108 at the distal end of the catheter 100 and the catheter 100 may be moved along the guide wire 5 such that the guide wire 5 may exit laterally from the lateral opening 108D of the lumen of the inner conduit 108. The guide wire 5 may be inserted into the body as is known in the art and advanced to the target region. The catheter 100 may be then pushed along the guide wire 5 and inserted into a body lumen or body passage (such as, for example, a blood vessel) as is known in the art. The rapid exchange catheter 100 also includes a handle 132 and a connecting conduit 114 for connecting the handle assembly 132 to the outer conduit 106. The connecting conduit 114 is preferably a flexible hollow tubular member made from a strong and flexible material such as, but not limited to, stainless steel in order to provide high pushability to the catheter 100. The connecting conduit 114 may be used to push the catheter 100 along the guide wire 5 to the target region. However, the connecting conduit 114 may also be made from other suitable materials having high pushability, such as for example, suitable reinforced polymer based materials including but not limited to, reinforced polyimide tubing or reinforced Nylon® tubing, and the like. The proximal end of the connecting conduit 114 is sealingly attached to the handle assembly 132 (by using any suitable attachment method known in the art, such as but not limited to a suitable glue or bonding compound or by thermal bonding and the like) and the distal end of the connecting conduit 114 is sealingly attached to the proximal end 106B of the outer conduit 106.

The handle 132 of the catheter 100 includes a hollow housing 136 having a space 138 therein. The housing 136 includes an inflation port 128 for fluidically connecting a standard indeflator device (not shown in FIG. 6) to the space 138. The indeflator device may provide inflation fluid through the space 138, the connecting conduit 114 and the space 126 for inflating the balloon 22. The connecting conduit 114 is attached to the housing 136 of the handle 132 such that the space 138 is in fluidic communication with the space 126 of the catheter 100 through the lumen of the connecting conduit 114. The handle 132 also includes a hydraulic accumulator 52 (as disclosed in detail hereinabove) and a closable stopcock 42. The hydraulic accumulator 52 is fluidically connected to the space 138 through the closable stopcock 42 such that when the closable stopcock 42 is closed, the hydraulic accumulator 52 is fluidically isolated from the space 138 and when the closable stopcock 42 is opened, the hydraulic accumulator 52 is fluidically in communication with the space 138.

The catheter 100 also includes a pulling member 134. The pulling member 134 may be a thin strong and flexible wire (having a circular cross section or a flattened elliptical cross section or the like) made from a suitable metal such as but not limited to stainless steel (but other types of different suitable materials, such as Kevlar® may also be used for implementing the pulling member 134).

A holding member 137 is attached to the first end of the pulling member 134. The holding member 137 may be used for pulling the pulling member 134 proximally (and for pushing the pulling member 134 distally, if necessary). The pulling member 134 passes into the space 138 through a sealed entry port 144 of the housing 136 and extends through the lumen of the connecting conduit 114 and through the space 126 formed between the outer conduit 106 and the inner conduit 108. Similar to the sealed entry port 44 of the catheter 50, the sealed entry port 144 allows longitudinal sliding movements of the pulling member 134 without leakage of any inflation fluid and without substantial pressure losses. The sealing may be achieved by using a suitable gasket(s) (not shown for the sake of clarity of illustration) or by any other suitable sealing method known in the art.

The distal end of the pulling member 134 is attached to the distal part 108A of the inner conduit 108. When the holding member 137 is pulled in the proximal direction, the distal part 108A of the inner conduit 108 moves proximally to shorten the sleeve member 108C in the longitudinal direction and to cause intussuscepting of the balloon 22 (as disclosed in detail hereinabove for the catheter 10).

It is noted that the catheters 10 and 30 illustrated in FIGS. 1-3 and 4 respectively may also be suitably modified to have a curved part of the inner conduit similar to the curved part 108B of the catheter 100. Such modified catheters may be operated in a way similar to the operation of the catheters 10 and 30 while enabling the guide wire 5 to exit laterally from the lateral side of the modified outer conduits of the catheters. Such modifications are well within the capacity of the person skilled in the art and may be easily understood and implemented without undue experimentation by suitably modifying the attachment points and structural configuration of the inner conduit and outer conduits of the catheters 10 and 30 while preserving the structure of the handle 12 of catheter 10 and the relief valve 42 of the catheter 30.

It will be appreciated by those skilled in the art that while the sleeve members of the catheters of the present application, such as, but not limited to the sleeve members 8C and 108C, are preferably corrugated sleeves (as illustrated in FIGS. 1-5 and 6) having a concertina-like or accordion—like shape, this is not obligatory and the catheters of the present application may use any type of variable length sleeve, such as but not limited to smooth (non-corrugated) cylindrical sleeve shapes, corrugated sleeve shapes having different types of corrugations (curved corrugations, corrugations having triangular cross-sections, corrugations having sinusoidally shaped cross sections, and the like), or other suitable sleeve shapes, as long as the flexible sleeve being used is capable of shortening (for example, as illustrated in the particular, non-limiting example of FIG. 2) in response to pulling the pulling member 24 or 34 or 134 proximally to enable the intussuscepting of the balloon 22.

For example, if a cylindrical, non-corrugated thin walled flexible sleeve is being used in the catheter instead of the corrugated sleeve 8C, the shortening of this part may be achieved by a crumpling of the flexible sleeve caused by buckling thereof due to the proximally directed force exerted on the walls of the sleeve by the distal part 8A of the inner conduit 8, resulting from pulling the plunger 18 proximally, with subsequent intussuscepting of the balloon 22. Such crumpling may cause the flexible sleeve to assume a tortuous and/or irregular crumples shape, causing the distance between the parts 8A and 8B to shorten (to allow the intussuscepting of the balloon 22) while still allowing inflation fluid to flow through the lumen of the tortuous/crumpled of the sleeve member 8C. Experiments performed with catheters using thin walled flexible cylindrical sleeve members have indicated that it is possible to achieve full intussuscepting of the balloon 22 while keeping the lumen of the crumpled twisted sleeve member open for fluid flow so that fluid ejected from the balloon 22 passes through the lumen of the shortened (crumpled) sleeve member and may be accommodated within the space 29 of the syringe-like member 18.

Moreover, while the generally preferred cross-sectional profile of the various parts of the inner conduit is a circular cross section, this in not obligatory and any one of the parts 8A, 8B and 8C and/or 108A, 108B and 108C may have other cross-sectional shapes, including but not limited too an elliptical cross-sectional shape, or any other suitable non-circular cross sectional shape.

It is further noted that while the particular shape of the balloon 22 illustrated in the drawings may be used in the rapid exchange catheters disclosed in the present application, this is not obligatory and other different balloon types and shapes may be used in the rapid exchange catheters of the present application. Thus, the rapid exchange catheters described herein may use, but are not limited to, any of the balloon types and shapes disclosed and illustrated in published international patent applications, publication numbers WO 2007/004221, and WO 2007/042935 and in international published applications WO 2010/001404 and WO 2010/001405, both of these PCT publications are incorporated herein by reference in their entirety for all purposes. For example, the balloons used in the catheters of the present application may include but are not limited to, a corrugated balloon, a balloon having at least one corrugated portion, a stepped balloon, a conical balloon, a distally tapering balloon, a distally and proximally tapering balloon a balloon having a non-uniform wall thickness, a balloon having a larger balloon wall thickness on its proximal portion and a balloon having a reinforced proximal portion.

Such additional balloon shapes usable with the rapid exchange catheters disclosed in the present application may include but are not limited to stepped balloons (as disclosed in WO 2010/001404), balloons having one or more corrugated parts (as disclosed in WO 2010/001405), stepped balloons having one or more corrugated parts, balloons having non-uniform wall thickness, balloons having a thickened proximal balloon part, balloons having a rounded distal end, balloons having one or more tapered parts, balloons having one or more conical parts, balloons having one or more frusto-conical parts, and the like.

Furthermore, the balloon used in the balloon catheters of the present application may include a balloon having in its inflated state, a shape which is capable of guiding the intussuscepting of the distal portion of the balloon upon proximal movement of the distal part of said inner conduit in relation to the outer conduit, or a balloon having, in its inflated state, a distal taper with a rounded distal extremity, or a balloon having, in its inflated state, a proximal taper with a rounded proximal extremity. The above balloon shapes and configurations are described in detail in published international patent applications WO 2007/004221 and WO 2007/042935.

Furthermore, while the catheters of the catheter systems described above are suitable for use with any suitable guide wire types as is known in the art, the catheters of the present application may also be used with any suitable embolization protection device (EPD) known in the art. For example, such EPD may include, inter alia, guide wire based EPDs including filters and/or distal occluding balloons, and/or proximal occluding balloons and/or baskets, and/or any other type of EPD known in the art and suitable for insertion through the lumen of the inner conduit 8 or 108 of the catheters. The internal diameter of the lumen of the inner conduits 8 and 108 should be selected such that it is large enough to accommodate and enable passage of the specific type of EPD used.

For example, the catheters of the present application may be used together with EPDs such as, but not limited to, the Guardwire® occlusion and aspiration system (commercially available from Medtronic Vascular, USA), the Spider-FX® embolic protection device, the (commercially available from ev3 Corporation, USA), the Abbot RX Accunet™ embolic protection device (commercially available from abbott Laboratories, USA), or any other suitable embolic protection device known in the art.

While the intussuscepting balloon catheters of the present application are quite efficient in trapping and removing plaque debris and or other particulate matter from the region of the treated target (such as for example particulate matter and debris resulting from plaque rupture during treatment or particulate matter released during patient treatment, such as but not limited to, opening of a stenosis during a PTCA procedure, an atherectomy and/or a stent deployment procedure (with or without the use of stenosis opening devices such as a rotablator, burring blade, an atherectomy laser or the like) and may effectively trap and remove a substantial amount of such potentially hazardous debris, the combined use of the catheters of the present application together with such various different EPD may further increase the total amount of debris and/or particulate material trapped and removed during treatment to even further reduce the risk of post-treatment patient embolization. In such a combined use of an intussuscepting catheter and an EPD, some debris or particulate matter which was not trapped by the intussuscepting balloon may be stopped and trapped by the EPD which may advantageously further decrease the total amount of debris released into the circulation during and after the procedure and may possibly even further decrease patient risk of embolization.

1. A rapid exchange balloon catheter comprising:
an outer conduit having a distal end and a proximal end;
a hollow inner conduit, suitable for passage over a guide wire, said inner conduit includes a distal part, a proximal part and a variable length sleeve member sealingly attached between said proximal part and said distal part of said inner conduit, said proximal part of said inner conduit is sealingly attached to the distal end of said outer conduit, said inner conduit is disposed within the lumen of said outer conduit such that the distal end of the distal part of said inner conduit extends beyond the distal end of said outer conduit;
a pulling member having a distal end attached to the distal part of said inner conduit and a proximal end disposed outside of said outer conduit, said pulling member is movably disposed within said catheter such that when said pulling member is moved proximally, the distal part of said inner conduit moves proximally to longitudinally shorten said sleeve member;
an inflatable balloon having a proximal balloon end sealingly attached to the outer surface of the distal end of said outer conduit, and a distal balloon end sealingly attached to the outer surface of the proximal part of said inner conduit, wherein the distal end of said balloon is capable of intussuscepting upon proximal movement of the distal part of said inner conduit in relation to said outer conduit; and
a fluid port for the introduction of an inflation fluid into the space formed between the inner surface of the outer conduit and the outer surface of the inner conduit and therefrom into the lumen of said balloon and for removal of said inflation fluid from said balloon.

2. The catheter according to claim 1, also including a pressure controlling mechanism for preventing or attenuating pressure changes within said balloon upon intussuscepting of said balloon.

3. The catheter according to claim 1, wherein said pressure controlling mechanism is selected from,
a syringe-like member in fluidic communication with said balloon, said syringe-like member includes a plunger member disposed therein, said plunger is attached to said pulling member, such that when said plunger member is pulled proximally, said pulling member moves proximally within said catheter to cause intussuscepting of said balloon wherein at least some of the inflation fluid ejected from said balloon is accommodated within a volume formed within said syringe like member upon proximal pulling of said plunger member,
a hydraulic accumulator in fluidic communication with said balloon,
a pressure relief valve in fluidic communication with said balloon, and any combinations thereof.

4. The catheter according to claim 3, wherein said pressure relief valve is an overpressure valve adapted to discharge inflation fluid whenever the pressure within said catheter exceeds a threshold pressure value.

5. The catheter according to claim 1, wherein said variable length sleeve member is selected from,
a corrugated sleeve like member,
a cylindrical sleeve like member having a circular cross section,
a sleeve like member having a non-circular cross section, and
a sleeve like member having at least one corrugated portion and at least one non-corrugated portion.

6. The catheter according to claim 1, wherein the length of said variable length sleeve member in the fully extended state is selected from,
a length smaller than half the length of said balloon when said balloon is fully extended,
a length equal to half the length of said balloon when said balloon is fully extended, and
a length larger than half the length of said balloon when said balloon is fully extended.

7. The catheter according to claim 1 wherein said balloon is selected from, a corrugated balloon, a balloon having at least one corrugated portion, a stepped balloon, a conical balloon, a distally tapering balloon, a distally and proximally tapering balloon, a balloon having a non-uniform wall thickness, a balloon having a larger balloon wall thickness on its proximal portion, a balloon having a reinforced proximal portion, a stepped balloon having one or more corrugated parts, a balloons having a thickened proximal balloon part, a balloon having a rounded distal end, a balloon having one or more tapered parts, a balloon having one or more conical parts, and a balloon having one or more frusto-conical parts.

8. The catheter according to claim 1, wherein the balloon is selected from,
a balloon having in its inflated state, a shape which is capable of guiding the intussuscepting of the distal portion of said balloon upon proximal movement of said distal part of said inner conduit in relation to the outer conduit,
a balloon having, in its inflated state, a distal taper with a rounded distal extremity, and
a balloon having, in its inflated state, a proximal taper with a rounded proximal extremity.

9. The catheter according to claim 1, wherein said proximal part of said inner conduit is selected from,
a straight proximal part sealingly attached to and opening at the proximal end of said outer conduit, and
a laterally curved proximal part sealingly attached to and opening at the lateral side of said outer conduit.

10. A method for collecting debris from an internal passage of a mammalian subject comprising the steps of:
a) inserting a rapid exchange balloon catheter as defined in claim 1 into said internal passage, and advancing said catheter until the distal end thereof has reached a target site, at which it is desired to collect debris;
b) inflating said balloon with inflation fluid;
c) moving said distal part of said inner conduit in a proximal direction, to cause intussuscepting of the distal end of said balloon;
d) deflating said balloon, to form therein a cavity into which debris is collected and entrapped; and
e) removing said balloon catheter from the internal passage of the subject, together with the entrapped debris.

11. The method according to claim 10, wherein said internal passage is a blood vessel.

12. The method according to claim 10, wherein said target site is in the vicinity of a stenosed portion of said blood vessel and wherein said method also includes the step of introducing through the lumen of said inner conduit a medical device for treating said stenosed portion.

13. The method according to claim 10, wherein said medical device is selected from a diagnostic device, a treatment device, and a combined treatment device and diagnostic device.

14. The method according to claim 13 wherein said treatment device is selected from an mechanical atherectomy device, a laser atherectomy device, an ultrasonic treatment device, an embolic protection device, a filter device, a basket-like device, a blade-like device, an occluding balloon device, an aspirating device and any combinations thereof.

15. The method according to claim 13 wherein said diagnostic device is selected from an imaging device, an infra-red imaging device, an imaging device operating in the visual range of the electromagnetic radiation spectrum, an ultrasonic imaging device, a magnetic resonance imaging device, an electrode based sensing device, a temperature sensing device, an electrochemical sensing device, a device for sensing the concentration of a chemical species and any combinations thereof.

16. The method according to claim 12 wherein said step of introducing a medical device also includes treating said blood vessel using said medical device.

17. The method according to claim 12 wherein said step of introducing a medical device also includes performing a diagnostic procedure on at least a portion of said blood vessel using said medical device.

18. The method according to claim 12, further including withdrawing said medical device outside of said catheter through the lumen of said inner conduit prior to performing said step (c).

19. The method according to claim 12, further including withdrawing said medical device into the lumen of said inner conduit prior to performing said step (e).

20. The method according to claim 10 and further including the step of introducing a stent into said body passage using said balloon.

21. The method according to claim 10 and further including the step of deploying said stent within said body passage during said inflating of said step (b).

22. A rapid exchange balloon catheter comprising:
an outer conduit having a distal end and a proximal end;
a hollow inner conduit, suitable for passage over a guide wire, said inner conduit includes a distal part, a proximal part and a variable length sleeve member sealingly attached between said proximal part and said distal part of said inner conduit, said proximal part of said inner conduit is sealingly attached to the distal end of said outer conduit, said inner conduit is disposed within the lumen of said outer conduit and positioned such that the distal end of the distal part of said inner conduit extends beyond the distal end of said outer conduit;
pulling/pushing means for proximally pulling and for distally pushing said distal part of said inner conduit within said outer conduit, such that when said pulling/pushing means is pulled proximally, the distal part of said inner conduit moves proximally to longitudinally shorten said sleeve member;
an inflatable balloon having a proximal balloon end sealingly attached to the outer surface of the distal end of said outer conduit, and a distal balloon end sealingly attached to the outer surface of the proximal part of said inner conduit, wherein the distal end of said balloon is capable of intussuscepting upon proximal movement of the distal part of said inner conduit in relation to said outer conduit; and
means for introducing an inflation fluid into said catheter and said balloon and for removal of said inflation fluid from said catheter and said balloon.
